(19) United States
(12) Patent Application Publication
(10) Pub. No.: US 2026/0014421 A1
(43) Pub. Date: Jan. 15, 2026

(54) APPARATUS, METHOD, AND COMPUTER PROGRAM PRODUCT FOR PROVIDING TRAINING GUIDANCE

(71) Applicant: Polar Electro Oy, KEMPELE (FI)

(72) Inventors: Jussi PELTONEN, KEMPELE (FI); Tommy GRANLUND, KEMPELE (FI)

(73) Assignee: Polar Electro Oy, KEMPELE (FI)

(21) Appl. No.: 18/841,711

(22) PCT Filed: Apr. 12, 2023
(86) PCT No.: PCT/FI2023/050206

§ 371 Date: Aug. 27, 2024

(30) Foreign Application Priority Data
Apr. 14, 2022 (EP) 22168347.7

(51) Int. Cl. A63B 24/00; A63B 71/06

(52) U.S. Cl. CPC A63B 24/0075; A63B 24/0062; A63B 71/0622; A63B 2220/30; A63B 2230/067

(57) ABSTRACT

An apparatus, method and computer program product for providing training guidance. The method comprises: acquiring training data that represents power output of a plurality of athletic performances of a user, the training data comprising first training data representing power output of a first athletic performance of a first duration and second training data representing power output of a second athletic performance of a second duration different from the first duration; fitting the training data into a decaying performance power curve representing power output as a function of duration of continuous athletic performance; determining, on the basis of the fitting, a set of training power output zones that conform to the performance power curve by decaying as a function of the duration; generating a training plan for a physical exercise on the basis of the set of training power output zones; and causing a user interface to output the training plan to the user.

### CROSS-REFERENCE TO RELATED APPLICATIONS

This application is a national phase application of International Application No. PCT/FI2023/050206, filed Apr. 12, 2023, which claims benefit and priority to European application Ser. No. 22/168,347.7, filed Apr. 14, 2022, which are incorporated by reference herein in their entireties.

### BACKGROUND

### Technical Field

Various embodiments relate to an apparatus, method, and computer program product for providing training guidance.

### SUMMARY

Sports performance testing is traditionally based on laboratory testing of athletes. However, laboratory testing is time-consuming and expensive, and the results may not translate well to real-world performance outside the laboratory. The laboratory testing may be used to find some user parameters such as the maximal oxygen uptake that is used for determining an aerobic threshold and an anaerobic threshold of the user. These thresholds are then used for determining personalized heart rate zones that guide the user's training. Alternatively, other training intensity and respective zones may be used, e.g. speed. An exercise may be based on exercising with a certain training intensity profile for a certain duration.

A mathematical model that describes running performance has been recently studied in Mulligan et al. (2018), A minimal power model for human running performance, PLOS ONE 13 (11): e0206645. The model may utilize real-world data, such as world record times, in contrast to data collected in a laboratory setting. Further study of the model has been performed in Emig & Peltonen, Human running performance from real-world big data, Nat Commun 11, 4936 (2020). However, practical applications of the model are currently very limited, and further development is needed.

According to an aspect, there is provided subject matter of independent claims. Dependent claims define some embodiments.

One or more examples of implementations are set forth in more detail in the accompanying drawings and the description of embodiments.

### BRIEF DESCRIPTION OF THE DRAWINGS

Some embodiments will now be described with reference to the accompanying drawings, in which

FIG. 1 illustrates embodiments of an apparatus for providing training guidance;

FIGS. 2A and 2B illustrate embodiments of a method for providing training guidance;

FIG. 3 and FIG. 4 illustrate embodiments of a performance power curve;

FIG. 5 and FIG. 6 illustrate embodiments related to providing training guidance;

FIG. 7 and FIG. 8 illustrate embodiments related to fitting the performance power curve;

FIG. 9 and FIG. 10 illustrate embodiments related to acquisition of time series data;

FIG. 11 illustrates embodiments for using and updating the performance power curve; and

FIG. 12 illustrates embodiments of an apparatus for providing training guidance as a block diagram.

### DETAILED DESCRIPTION

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may contain also features/structures that have not been specifically mentioned.

Reference numbers, both in the description of the embodiments and in the claims, serve to illustrate the embodiments with reference to the drawings, without limiting it to these examples only.

The embodiments and features, if any, disclosed in the following description that do not fall under the scope of the independent claims are to be interpreted as examples useful for understanding various embodiments of the invention.

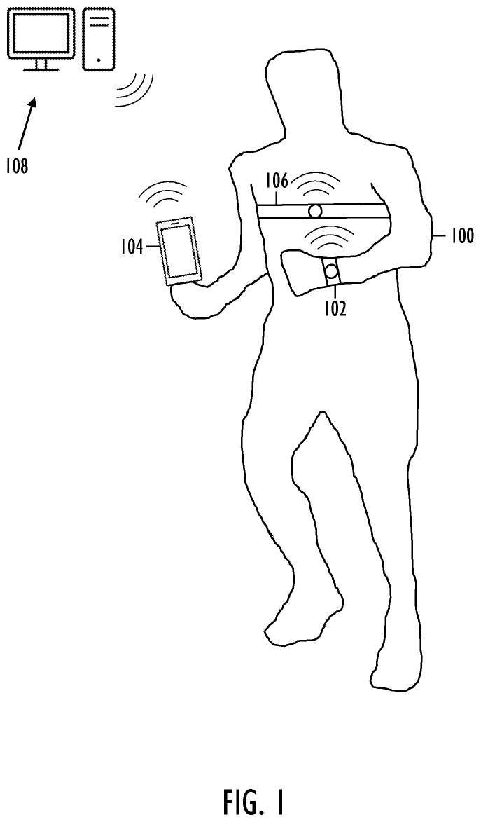

FIG. 1 illustrates embodiments of an apparatus for providing training guidance to a user 100. The user 100 is illustrated to be performing a physical exercise or an athletic performance. The physical exercise may be running (such as in FIG. 1), cycling, swimming, cross-country skiing, rowing, or any other sport for which power output may be directly or indirectly measured. The power output may refer to power generated or output by the user during the athletic performance.

The apparatus of FIG. 1 may be or comprise a wearable device 102, a portable electronic device 104, a wearable sensor device 106, and/or a server computer 108. The apparatus may be a single physical apparatus or a distributed apparatus (a system). For example, the wearable device 102 may operate as the single physical apparatus. The distributed apparatus or system may comprise a plurality of communicatively coupled physical devices such as the wearable device 102, the wearable sensor device 106, the portable electronic device 104, and/or the server computer 108.

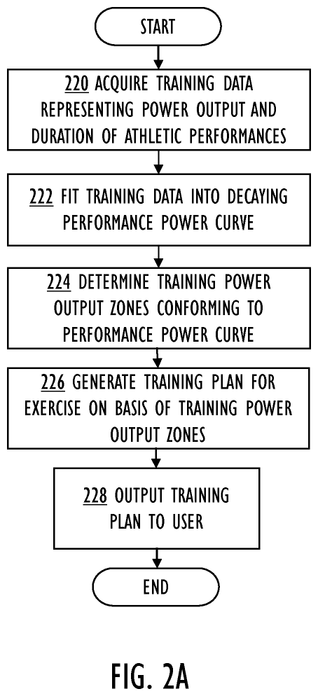

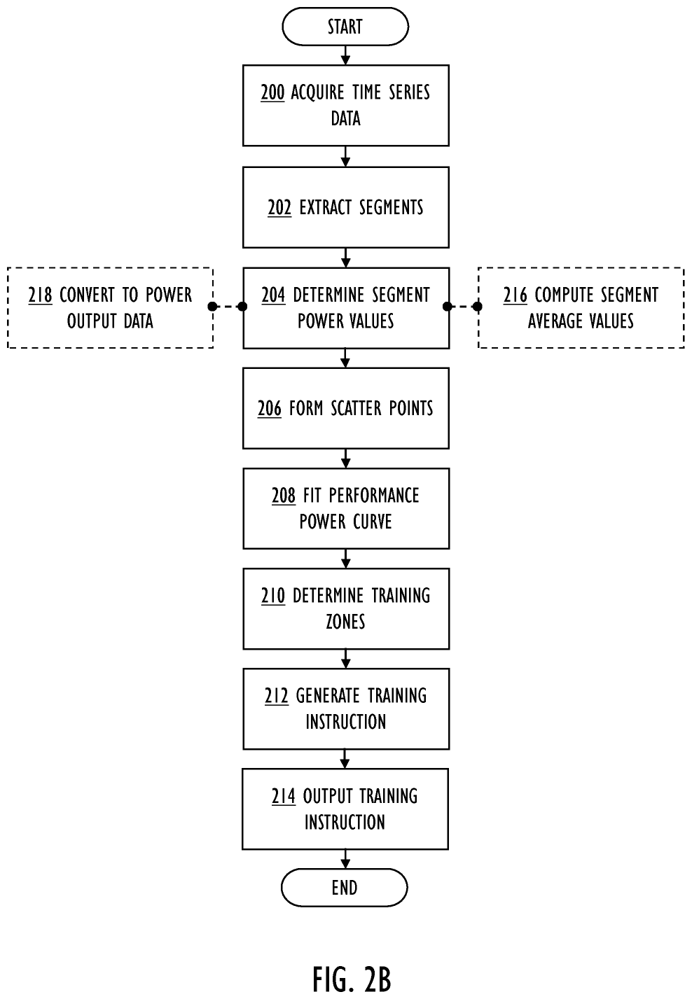

The apparatus may be configured to perform a computer-implemented method illustrated in FIG. 2A. Referring to FIG. 2A, the method comprises: acquiring 220 training data that represents power output of a plurality of athletic performances of the user 100, the training data comprising first training data representing power output of a first athletic performance of a first duration and second training data representing power output of a second athletic performance of a second duration different from the first duration; fitting 222 the training data into a decaying performance power curve representing power output as a function of duration of continuous athletic performance; determining 224, on the basis of the fitting, a set of training power output zones that conform to the performance power curve by decaying as a function of the duration; generating 226 a training plan for a physical exercise on the basis of the set of training power output zones; and causing a user interface to output the training plan to the user.

The fitting may be performed and the resulting performance power curve computed as described in Mulligan et al mentioned in Background. In the following, some embodiments of the computation of the performance power curve are described.

The performance power curve may thus define the shape of the training power output zones. The performance power curve and the training power output zones may be defined in terms of an average power as a function of the duration of an athletic performance. The exercise may refer to a main work period of a physical exercise or a race.

FIG. 2B illustrates another aspect of the method for improved training guidance. The method of comprises: acquiring 200 time series data that represents power output of athletic performances by the user; extracting 202 a plurality of segments from the time series data, wherein the plurality of segments comprise a first segment having a first duration and a second segment having a second duration, the second duration being different from the first duration; determining 204 segment power values on the basis of the time series data of the extracted plurality of segments; forming 206 a set of scatter points describing the segment power values as a function of respective durations of the extracted plurality of segments, and fitting 208 a performance power curve for the set of scatter points; determining 210 a set of training zones on the basis of the performance power curve; generating 212 a training instruction on the basis of the set of training zones; and causing 214 a user interface to output the training instruction.

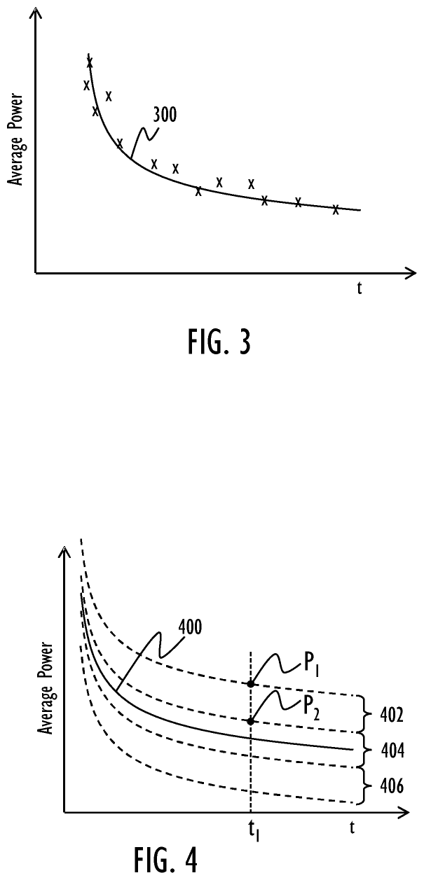

The solutions described herein provide individualized training guidance to the user. The performance power curve and the training zones derived from it characterize the user's athletic performance. By generating the training instruction on the basis of the set of training zones, the user may receive individualized feedback and guidance on their training. The performance power curve aims to present how the user's capability to sustain a certain average power output for a certain duration. As illustrated in the performance power curve of FIG. 3, the user is capable of sustaining a greater average power output when the duration of the athletic performance is short. It is generally known that one can sustain a greater constant speed for only a short duration and a slow speed for a long duration. However, this characteristic has not been brought into the training zones in the personalized manner the present invention does. The training zone and the training power output zone refer to the same zone in this description. The training zone refers to a zone bound by an upper limit and a lower limit that is defined in terms of power output of the user. As described herein, the power output may be quantized as heart rate or speed or velocity, for example. The time series data represents power output of multiple athletic performances performed by the user, each athletic performance having a specific duration (t). The athletic performance may be understood as a single physical exercise, a race or a competition, or a part of the physical exercise. Power output of the athletic performances may be directly measured by power sensors, commonly used with (stationary) bicycles, for example. In an embodiment, the time series data is power data measured by a power sensor. Power output may also be derived from other kinds of time series data. It has been found that linear relationships exist between power, velocity, and heart rate. These linear relationships may be exploited to convert e.g. velocity data and/or heart rate data to power data, or vice versa. Further, the apparatus may derive velocity data from e.g. location data measured by a location sensor, and/or the apparatus may derive heart rate data from electrocardiogram (ECG) data measured by an ECG sensor, for example.

The time series data may be time series data of past athletic performances that is acquired from one or more memories, and/or time series data acquired in real time by the apparatus. The apparatus may comprise or be coupled to one or more sensors configured to measure the time series data, and the apparatus may be configured to acquire the time series data from the one or more sensors. The acquiring may also comprise receiving, over a wired or wireless 15 connection, the time series data from one or more sensors or one or more memories.

The time series data is an embodiment of the training data.

The training data may be a collection of samples where each sample has a power value representing the power output and a duration value representing duration of the respective athletic performance. FIG. 3 illustrates such a set of samples (called scatter points below). The power value may be an average speed counted over the respective duration. In the simplest form, the apparatus may acquire as user inputs from the user interface or from the memory the respective information to generate the set of samples. Such information needed per sample 25 may comprise, for example, a distance and duration needed to cross the distance of the athletic performance. The average speed may then be computed by the ratio of the distance and the duration to get the power value. In order to do the fitting properly, at least two samples of athletic performances having different durations are required. Preferably more than two samples are acquired, with a substantially 30 wide span of durations to improve the accuracy of the fitting.

From another perspective, the training data may comprise the time series data. The time series data may be understood as a collection of measurement data measured during one or more athletic performances. The training data may comprise measurement data of at least one athletic performance stored in the at least one memory, and the at least one processor is configured to compute a power output of the athletic performance on the basis of the measurement data and use the power output as the training data.

In an embodiment, the apparatus is configured to extract 202 a plurality of segments from the time series data. The plurality of segments comprise a first segment having a first duration and a second segment having a second duration, and the second duration is different from the first duration. A segment may be a physical exercise, a race, or a part of the physical exercise. Preferably, the segments are taken from different exercises or races. In principle, at least two different durations may be required to fit the performance power curve. Alternatively or additionally, durations of the segments may be examined via distances travelled during the segments. For example, when the time series data comprises location data, the apparatus may determine a distance travelled by the user on the basis of the location data. The apparatus may select the segments to be extracted on the basis of an assumption that different distances are generally travelled in different durations of time. The first segment may thus have a first distance and the second segment may have a second distance, and the second distance is different from the first distance.

The performance power curve and the associated training zones are particularly designed for running and to guide the user in running exercises. As a consequence, the training data may comprise or consist of training data from past running exercises or running races. However, the same principles may be directly applicable to other sports, such as racewalking that is very similar to the running. The same principles may be applied to other sports as well such as cycling, swimming or cross-country skiing, considering that a metric of an average training power output target determined on the basis of the training zones (a training zone selected for the exercise) is selected appropriately. For example, speed is a suitable training power output target metric for running but less suitable for cycling or skiing. In cycling, pedalling power may be a suitable metric. In any case, the training data is preferably selected only from the exactly same type of sports type, such as the running. Mixing different sports types into the same performance power curve can be implemented but it may be prone to inaccurate estimation of the user's capabilities.

In an embodiment, the training data represents maximum power output of the user in the respective athletic performances, such as the exercise(s) or race(s). An example of the maximum power output includes the user's best performance (record average power output) in an athletic performance. The training data may thus comprise or consist of the user's record times and respective distances for various races/exercises of different distances, e.g. a record for 5 km, a record for 10 km, a record of half marathon, and a record of marathon. These examples result in a performance power curve that is based on the user's maximum physiological capabilities. Another alternative is to use training data that represents the maximum level at which the user desires to exercise, representing a comfortable maximum power output. The user may input this information manually to the apparatus, or the apparatus may harvest this information from the user's past exercises stored in the memory of the apparatus. In this case, the performance power curve is adapted to the level where the user wishes to train. In order to select the suitable training data, the user may indicate whether the performance power curve shall be based on the user's maximum physiological capabilities (the records) or on the user's comfortable maximum performances. The user may handpick or approve the training data to be included in the fitting.

The apparatus may apply some filters to decide whether or not a certain information is eligible for the training data. For example, the maximum performance eligible to the performance power curve shall not be older than a specified time period, e.g. only maximum performances made within one year are eligible. In case there is not a sufficient amount of recent training data available, the apparatus may notify the user of this and ask the user to provide more training data (e.g. carry out an exercise of a certain distance). Alternatively, the apparatus may notify the user that the fitting may be inaccurate because of the old training data.

As described herein, even a part of a past athletic performance may be considered eligible as the training data. For example, if a running exercise includes a work period where the user has made the best maximum performance over a certain distance and during a work period forming a subset of work periods of the running exercise, the distance and duration of that work period may be taken to the training data. In such an embodiment, the apparatus may identify a continuous time interval of the athletic performance that is associated with a power output greater than an inclusion threshold (the maximum performance), and select the power output and the duration of the continuous time interval for the fitting and exclude a power output of at least one other time interval of the first athletic performance from the fitting. The inclusion threshold may be set so that it represents the user's best performance in the particular exercise (at an exercise race/exercise with a particular distance).

The apparatus is configured to determine 204 segment power values on the basis of the time series data of the extracted plurality of segments. In an embodiment, the apparatus is configured to compute 216 segment average values from the time series data of the extracted plurality of segments to determine the segment power values. The segment power values may therefore be segment average power values. The apparatus may compute an average value for each segment from the time series (measurement) data of the respective segment. Average values have been observed to sufficiently characterize the user's performance, and are computationally efficient compared to some other statistical parameters, for example.

In an embodiment, the time series data comprises velocity data and/or heart rate data, and the apparatus is configured to convert 218 the velocity data and/or the heart rate data to power output data, and to determine the segment power values on the basis of the power output data. In the context of the described embodiments, speed and velocity data are technically equivalent because the absolute directions of the user's motion are not of interest. As a consequence, the speed and velocity are used here inter-changeably. When the apparatus is configured to compute segment average values, the average values may be computed before or after the conversion to power output data.

When the time series data comprises heart rate data, the apparatus may perform a heart rate-power conversion on the heart rate data to obtain the power output data. The heart rate-power conversion may be a transformation of the form P=A (HR)+b, wherein P represents power, HR represents heart rate, A represents a linear mapping coefficient, and b represents a translation parameter. The translation parameter b may be zero.

Heart rate is linearly proportional to power at least under certain conditions. The heart rate of the user and power output by the user are linearly proportional at least when the user is performing aerobic exercise, in contrast with anaerobic exercise. The phenomenon of heart rate drift may also distort the relationship between heart rate and power. Heart rate drift is observed when heart rate increases despite a constant power output. A rapid change in exercise intensity may result in a large heart rate drift. A decrease in blood volume, an increase in core temperature, and neuromuscular fatigue may result in a gradual heart rate drift. The apparatus may be configured to detect, on the basis of the time series data, heart rate drift, or if the user is performing anaerobic exercise. If neither heart rate drift is detected nor is the user performing anaerobic exercise, the apparatus may perform the heart rate-power conversion on the heart rate data to obtain the power output data.

When the time series data comprises velocity data, the apparatus may perform a velocity-power conversion on the velocity data to obtain the power output data. The velocity-power conversion may be a transformation of the form P═Cv+d, wherein P represents power, v represents velocity, C represents a linear mapping coefficient, and d represents a translation parameter. The translation parameter d may be zero.

Velocity is directly proportional to power in some sports such as running, but not in others such as cycling. The apparatus may be configured to detect, on the basis of the time series data, the sport of the athletic performance performed by the user. Alternatively or additionally, the apparatus may receive the sport from the user as an input via the user interface. The apparatus may compare the detected sport to a list of sports stored e.g. in a memory of the apparatus, for which sports velocity is proportional to power. If the sport is included in the list, the apparatus may perform the velocity-power conversion on the velocity data to obtain the power output data.

The apparatus may utilize multimodal conversions, such as Running Power by Polar, to obtain the power output data. The apparatus may compute Running Power from velocity and gradient time series data obtained from global positioning system (GPS) and barometric sensors, resulting in power output data. The apparatus may determine the segment power values on the basis of the power output data.

The apparatus is configured to form 206 a set of scatter points describing the segment power values as a function of respective durations of the extracted plurality of segments. The apparatus may obtain durations of the extracted plurality of segments from timestamps or metadata of the time series data, for example. The apparatus is further configured to fit 208 a performance power curve for the set of scatter points.

FIG. 3 illustrates embodiments of the set of scatter points and the performance power curve 300. In FIG. 3, the scatter points are plotted in a coordinate system wherein the vertical axis represents average power, and the horizontal axis represents duration. In FIG. 3, the segment power values are segment average power values. The performance power curve 300 is fitted to the set of scatter points using e.g. the method of least squares. Because it is known from the science that the performance power curve is a decaying curve, particularly exponentially decaying curve (ex), a base curve may be provided to which the fitting is performed. The base curve may be based on function e-x or on a more complex function better approximating the general shape of the average performance power curve, for example. It should be appreciated that each user has a unique performance power curve although a generic shape of the performance power curves of different users is similar. For example, the average power each user is capable of outputting for a long duration varies, depending on the user's endurance characteristics. Also, the degree of decay may vary between users. As described above, velocity or speed may be directly proportional to power in some conditions. The following paragraphs describe a mathematical model, also known as a universal performance model, in terms of speed. The model expresses exercise intensity on a relative power scale, which varies between zero corresponding to basal metabolic rate, and unity at maximal aerobic power (MAP) generation. Alternatively or additionally, the model may be represented in terms of power or heart rate, with the help of e.g. conversions similar to those described above.

The relation between a fastest performance time T and distance d covered in that time Tis characterized by two equations (1) and (2) shown below. Equation (1) is applicable when d>=d_{c}, and equation (2) is applicable when d<=d_{c}, wherein d_{c} is a crossover distance given by d_{c}=t_{c}v_{c}, wherein t_{c} and v_{c} are crossover time and speed. The crossover speed corresponds to speed or velocity at maximal oxygen consumption (VO2max), and crossover distance is a distance that is travelled in time t_{c} with speed v_{c}. Parameter v_{c} coincides with a speed at which VO2max can be achieved and therefore it is also known as maximal aerobic speed (MAS). MAS is an example of maximal aerobic power (MAP) that is the highest power the user can output. It has been estimated that t_{c} may be 5 to 7 minutes. In an embodiment, t_{c} is 6 minutes.

$$T ( d ) = \frac{{t}_{c}}{{γ}_{l}} \frac{d}{{d}_{c}} \frac{1}{{W}_{- 1} [ \frac{d}{{d}_{c}} \frac{exp {(}^{- 1} {/}_{{γ}_{l}} )}{{γ}_{l}} ]} \qquad ( 1 )$$

$$T ( d ) = \frac{{t}_{c}}{{γ}_{s}} \frac{d}{{d}_{c}} \frac{1}{{W}_{- 1} [ \frac{d}{{d}_{c}} \frac{exp {(}^{- 1} {/}_{{γ}_{s}} )}{{γ}_{s}} ]} \qquad ( 2 )$$

Parameters γₛ and γ₁ rare related to endurance parameters Eₛ and Eₗ. Long distance endurance parameter Eₗ is given by the relation Eₗ=exp(0.1/γ₁), and short distance endurance parameter Eₛ is given by the relation Eₛ=exp(0.1/γₛ). The subscripts/and s refer to long and short distance performance, respectively. W-1 is real branch of the Lambert W-function, which is defined as the multi-valued inverse of the function w→w exp(w).

Eₗ is also known as Endurance index, i.e. a ratio of a duration for which an athlete is able to maintain 90% of MAS and a duration for which the athlete is able to maintain MAS. For example, if an athlete can maintain 90% of MAS for 18 minutes, and MAS for 6 minutes, the athlete's endurance index is 18/6=3.

The model described above may be utilized to acquire a threshold power curve. In an embodiment, the performance power curve is the threshold power curve. The performance power curve may be a power law or a broken power law. Even though the performance power curve is described herein in the form of a power vs. duration function, power and/or duration may be replaced by other parameters whose relationship with power is known or may be estimated. For example, power may be replaced by heart rate or speed or velocity, and/or duration may be replaced by distance. The apparatus may determine the relationship between duration and distance using velocity data and a formula d=tv, wherein dis distance, tis duration, and v is velocity. Further, the variables power and duration (and/or their replacements) may be switched around so that the scatter points describe the durations of the extracted plurality of segments as a function of segment power values, for example.

Training zones have been previously based on the idea of “threshold power” that describes power that muscles can supply for unlimited amount of time. This time-invariant threshold power has led to static training zones that are also time-invariant. However, this defies observations as no power can be sustained without limitations. Analysis of running world records have revealed that maximal power that runner can sustain decreases rapidly over first 12 minutes and then continues to drop indefinitely at a slower rate. The performance power curve personalized to the user in the above-described manner describes this characteristic of the user, and the performance power curve is used to generate training zones that are variant according to duration, wherein the varying nature refers to that the training zones are defined differently for physical exercises of different durations.

The apparatus is configured to determine 210, 224 a set of training zones on the basis of the performance power curve. Determining the set of training zones on the basis of the performance power curve may refer to determining the set of training zones on the basis the curve itself (e.g. the shape and/or values of the curve), and/or on the basis of one or more parameters characterizing the curve. FIG. 4 illustrates a performance power curve 400 in an average power vs. duration coordinate system. Each training zone 402 to 406 has a decaying shape that conforms to the decaying shape of the performance power curve. Each training zone may define an (average) power range for each duration t of a physical exercise or a part of the physical exercise. The part may refer to the main part of the exercise, e.g. it may exclude a warm-up cooldown periods, if provided in the exercise. In FIG. 4, three training zones 402, 404, 406 have been determined on the basis of the performance power curve. Training zone 402 includes power values above the performance power curve, training zone 404 includes power values equal to, above, and below the performance power curve, and training zone 406 includes power values below the performance power curve. The training zones may be non-overlapping as illustrated in FIG. 4.

FIG. 4 illustrates the performance power curve on the training zone 404 that has at least one training zone 402 above it. In this case, the performance power curve may not be adapted to a level below the user's physiological maximum performance (the maximum comfortable level described above). In another embodiment where the maximum performances are used for generating the performance power curve, the maximum power output zone 402 may be arranged around the performance power curve. In order to know the training zone to which the performance power curve belongs, the apparatus may receive as user input information on whether the performance power curve is based on physiological maximum performance (the maximum power output zone 402 around the performance power curve) or on a lower performance level (another training zone around the performance power curve).

In an embodiment, the apparatus is configured to determine a training zone 402, 404, 406 of the set of training zones by multiplying the performance power curve 400 by a constant multiplier. This way, information on the relationship between power and duration contained in the performance power curve may be efficiently transferred to the set of training zones, and the training zones may closely follow the shape of the performance power curve. The apparatus may generate a boundary for the training zone by multiplying the performance power curve 400 by the constant multiplier. In an embodiment, each training zone 402, 404, 406 of the set of training zones is defined by the performance power curve 400 multiplied by a constant multiplier. The constant multiplier for each training zone may be different, i.e. zone-specific.

As an example of some of the above embodiments, three training zones may be defined as follows. The training zones may be somewhat analogous to the conventional training zones in terms of how each zone characterizes an exertion level (power output) the user exhibits. A first training zone, a “recovery” training zone 406, may be defined with the performance power curve multiplied by 0.95 as an upper bound. A second training zone, a “productive” training zone, may be defined with the performance power curve multiplied by 0.95 as a lower bound, and the performance power curve multiplied by 1.05 as an upper bound. A third training zone, a “maximal” training zone, may be defined with the performance power curve multiplied by 1.05 as a lower bound. The training zones are not necessarily completely bounded; the first training zone may not have a lower bound, and/or the third or last training zone may not have an upper bound.

In an embodiment, the constant multiplier is between 0 and 1. As an example, three training zones may be defined in the order of increasing power as follows: A first training zone, a “recovery” training zone, may be defined with the performance power curve multiplied by 0.5 as a lower bound and the performance power curve multiplied by 0.7 as an upper bound. A second training zone, a “productive” training zone, may be defined with the performance power curve multiplied by 0.7 as a lower bound and the performance power curve multiplied by 0.9 as an upper bound. A third training zone, a “maximal” training zone, may be defined with the performance power curve multiplied by 0.9 as a lower bound. In an embodiment, the training zones are completely bounded (in terms of power). Considering again the above example, the third training zone may have an upper bound of the performance power curve multiplied by 1.0 or even 1.1, resulting in three completely bounded training zones.

It is also noted that the number of training zones in the set of training zones is not limited to three but may be any suitable number, such as 2, 3, 4, or 5, for example.

The apparatus is configured to generate 212 a training instruction on the basis of the set of training zones. The training instruction may guide the user in a physical exercise or exercises to reach the desired training effect. The training instruction may be based on selecting the training zone on the basis of the desired training effect of the exercise and then selecting the duration and/or the average power for the exercise within that training zone. The apparatus is configured to cause 214 a user interface to output the training instruction to the user 100.

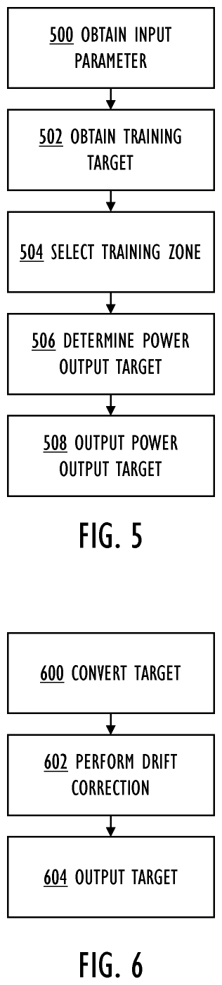

FIG. 5 illustrates an embodiment comprising obtaining 500, 502 an input parameter and a training target for the physical exercise (a training session); selecting 504, on the basis of the training target, a training zone from the set of training zones for the exercise; determining 506 a power output target on the basis of the input parameter and the selected training zone; and causing 508 the user interface to output the power output target for the exercise. In an embodiment, the input parameter is a duration parameter or a distance parameter. The training target may be a qualitative target describing the training effect or training benefit sought from the exercise, such as “recovery”, “development”, or “race” (equivalent to maximum performance) describing a goal of the training session or part of the training session, for example. The apparatus selects a training zone mapped to the training target from the set of training zones. A mapping of training targets and training zones may be stored in a memory of the apparatus, e.g. the target “recovery” may correspond to a recovery training zone, a “development” target may correspond to a productive training zone, and a “race” target may correspond to a maximal training zone. The apparatus may be configured to select the training zone for the training session using the mapping. The apparatus selects a power output target on the basis of the input parameter and the selected training zone. FIG. 4 illustrates an example wherein the input parameter is a duration parameter t₁, the training target is “race”, and the selected training zone is maximal training zone 402 mapped to the training target “race”. The apparatus may determine power values P₁ and P₂ that correspond to the upper and lower bounds of the training zone at duration t₁. The apparatus may determine the power output target as a range, such as [P₁, P₂], or as a power value, such as the average of P₁ and P₂. For example, the power output target may be defined in terms of a speed range or an average speed value mapped to the input duration t₁. In other words, the user may input that he/she wants to exercise at the maximum effort for a specific duration, and the apparatus then uses the maximum effort training zone to find an average speed or speed range specific for that duration to train the maximum effort. As illustrated in FIG. 4, the speed may be greater for shorter durations of the exercise than and decrease for the greater durations of the exercise. In other words, the maximum training effort is reached with different speeds or speed ranges for different durations of the exercise. Alternative to the duration as the input parameter, the user may input the distance he/she wishes to cover in the training session. Again, the apparatus may compute the average speed or speed range mapped to the selected training zone and the input distance parameter. The apparatus causes the user interface to output the power output target to the user as e.g. a value or a range of values, in Watts, for example. Since Watts is abstract to an average user, the speed, or heart rate may be used as an alternative.

Table below illustrates an example of the illustration of the training zones where the training zones are provided with finer granularity than in FIG. 4. The training zones are rather provided as a scale of the training effect. Furthermore, the user may provide, as the input parameter, either distance or time of the exercise and then evaluate the different average times and speeds and respective training effects.

| Training | Speed | Time | | Power |
|---|---|---|---|---|
| effect | (km/h) | per km | Time | (% MAP) |
| 100 | 12.1 | 0.04.58 | 0.30.00 | 67% |
| 98 | 11.8 | 0.05.05 | 0.30.37 | 65% |
| 96 | 11.6 | 0.05.11 | 0.31.15 | 64% |
| 94 | 11.3 | 0.05.18 | 0.31.55 | 63% |
| 92 | 11.1 | 0.05.24 | 0.32.37 | 61% |
| 90 | 10.9 | 0.05.32 | 0.33.20 | 60% |
| 88 | 10.6 | 0.05.39 | 0.34.05 | 59% |
| 86 | 10.4 | 0.05.47 | 0.34.53 | 57% |
| 84 | 10.1 | 0.05.55 | 0.35.43 | 56% |
| 82 | 9.9 | 0.06.04 | 0.36.35 | 55% |
| 80 | 9.6 | 0.06.13 | 0.37.30 | 53% |
| 78 | 9.4 | 0.06.23 | 0.38.28 | 52% |
| 76 | 9.2 | 0.06.33 | 0.39.28 | 51% |
| 74 | 8.9 | 0.06.43 | 0.40.32 | 49% |
| 72 | 8.7 | 0.06.55 | 0.41.40 | 48% |
| 70 | 8.4 | 0.07.06 | 0.42.51 | 47% |

% MAP refers to a percentage of the maximal aerobic power. The Table above is based on the target duration of 30 minutes where the user reaches the maximum effort (training effect 100) for the 30-minute exercise with an average speed 12.1 km/h (4 minutes 58 seconds per kilometre). Now, if the user selects the training effect 90, for example, the apparatus may set the target speed 10.9 km/h as the power output target for the exercise and guide the user to reach or maintain that speed during the exercise. The same user having the same performance power curve entering the 60 minutes target duration as the input parameter may then get the power output target from the following Table.

| Training | Speed | Time | | Power |
|---|---|---|---|---|
| effect | (km/h) | per km | Time | (% MAP) |
| 100 | 11.1 | 0.05.23 | 1.00.00 | 62% |
| 98 | 10.9 | 0.05.30 | 1.01.13 | 60% |
| 96 | 10.7 | 0.05.37 | 1.02.30 | 59% |
| 94 | 10.5 | 0.05.44 | 1.03.50 | 58% |
| 92 | 10.2 | 0.05.52 | 1.05.13 | 57% |
| 90 | 10.0 | 0.05.59 | 1.06.40 | 55% |
| 88 | 9.8 | 0.06.08 | 1.08.11 | 54% |
| 86 | 9.6 | 0.06.16 | 1.09.46 | 53% |
| 84 | 9.3 | 0.06.25 | 1.11.26 | 52% |
| 82 | 9.1 | 0.06.34 | 1.13.10 | 50% |
| 80 | 8.9 | 0.06.44 | 1.15.00 | 49% |
| 78 | 8.7 | 0.06.55 | 1.16.55 | 48% |
| 76 | 8.5 | 0.07.06 | 1.18.57 | 47% |
| 74 | 8.2 | 0.07.17 | 1.21.05 | 46% |
| 72 | 8.0 | 0.07.29 | 1.23.20 | 44% |
| 70 | 7.8 | 0.07.42 | 1.25.43 | 43% |

The Table below illustrates yet another example for the same user and the same performance power curve as in the Tables above, where the user now enters the desired distance (3 kilometres in this example). In response to this input parameter, the apparatus may provide the power output target from the following Table, according to the desired training effect.

| Training | Speed | Time | | Power |
|---|---|---|---|---|
| effect | (km/h) | per km | Time | (% MAP) |
| 100 | 13.3 | 0.04.31 | 0.13.33 | 73% |
| 98 | 13.0 | 0.04.36 | 0.13.49 | 72% |
| 96 | 12.8 | 0.04.42 | 0.14.07 | 71% |
| 94 | 12.5 | 0.04.48 | 0.14.25 | 69% |
| 92 | 12.2 | 0.04.54 | 0.14.43 | 68% |
| 90 | 12.0 | 0.05.01 | 0.15.03 | 66% |
| 88 | 11.7 | 0.05.08 | 0.15.24 | 65% |
| 86 | 11.4 | 0.05.15 | 0.15.45 | 63% |
| 84 | 11.2 | 0.05.23 | 0.16.08 | 62% |
| 82 | 10.9 | 0.05.30 | 0.16.31 | 60% |
| 80 | 10.6 | 0.05.39 | 0.16.56 | 59% |
| 78 | 10.4 | 0.05.47 | 0.17.22 | 57% |
| 76 | 10.1 | 0.05.56 | 0.17.49 | 56% |
| 74 | 9.8 | 0.06.06 | 0.18.18 | 54% |
| 72 | 9.6 | 0.06.16 | 0.18.49 | 53% |
| 70 | 9.3 | 0.06.27 | 0.19.21 | 51% |

It should be appreciated that the Tables above are just to illustrate how the performance power curve and the respective training zones are translated into the power output target(s) for the exercise. The actual implementation and the user interfacing may vary.

In the embodiments above, the power output target may represent the average power output of the exercise. This is directly applicable to a steady-pace exercise, for example, where the user is instructed to maintain the selected speed representing the power output target. In case the user exceeds the selected speed, the apparatus may instruct the user to reduce speed and, in case the user's speed is below the selected speed, the apparatus may instruct the user to increase the speed. In other exercises that is not designed to be performed on a steady pace or steady power output, the performance power curve may still be usable. Let us consider an interval exercise comprising several work periods and a rest period between the consecutive work periods. If a certain training zone is selected on the basis of the desired training effect, the apparatus may use that training zone directly in a steady-pace exercise but scale down a certain level for the purpose of the interval exercise. Therefore, the selected exercise type may be yet another input to the apparatus. Let us remind that in the interval exercise typically the user cannot exhibit the maximum performance in any of the work periods. Therefore, it may be feasible to scale down the power output target and use the lower power output target for the work periods of the interval exercise.

The apparatus may obtain the input parameter(s) and/or the training target from the user as a user input via the user interface. Alternatively or additionally, the apparatus may read the input parameter and/or the training target from a memory of the apparatus, e.g. from a training plan designed for the user beforehand, and/or the apparatus may receive the input parameter and/or the training target over a wireless connection from another apparatus, e.g. a server computer.

FIG. 6 illustrates embodiments related to the power output target. In an embodiment, the apparatus is configured to convert 600 the power output target to a heart rate target and/or a velocity target, and to cause 604 the user interface to output the heart rate target and/or the velocity target as the power output target. Power may be difficult to understand especially by inexperienced users, and conversion to heart rate or velocity may make the target better understandable to the users. Further, power data may be more difficult to acquire than velocity or heart rate data, and comparison of the target to acquired velocity and/or heart rate data is thus facilitated. The velocity-power conversion and/or the heart rate-power conversion described earlier, or another conversion, may be utilized to convert the power output target to a heart rate target and/or a velocity target.

In an embodiment, the apparatus is configured to convert the power output target to a heart rate target and to perform 602 a drift correction on the heart rate target, and to cause the user interface to output the corrected heart rate target as the power output target. Heart rate drift, as described earlier, corresponds to an increase in heart rate despite constant power output. When the power output target is output as a heart rate target, the heart rate target may be incorrect or even unachievable if the user is experiencing heart rate drift. The heart rate target may thus be corrected using a drift correction to adjust, typically increase, the heart rate target to one that more accurately represents the desired power output target. The drift correction may be based on heart rate demand, which represents a true heart rate that user's effort requires. When the relation between heart rate and heart rate demand is known, the user may be instructed to achieve a suitable heart rate even in the case of heart rate drift. A model describing the relation between heart rate and heart rate demand is described in Stirling, James Robert, et al., A Model of Heart Rate Kinetics in Response to Exercise. Journal of Nonlinear Mathematical Physics, October 2008, Volume 15, Supplement 3, pages 426-436.

The apparatus may be further configured to acquire power data, velocity data, and/or heart rate data of the user. The acquiring may be performed similarly to that of the time series data. The apparatus may be configured to cause the user interface to output the acquired power data, velocity data, and/or heart rate data. The apparatus may be configured to compare the power data to the power output target, to compare the velocity data to the velocity target, and/or to compare the heart rate data to the heart rate target. If the power/velocity/heart rate data does not meet or fulfil the corresponding target, the apparatus may be configured to cause the user interface to output an indication to the user. Meeting the target herein may refer to the data falling within the target range, for example. The indication may be a graphical indication displayed on a display screen or another display unit, an audio indication output by an audio output device such as a loudspeaker, or a haptic indication output by a haptic output device configured to provide haptic indications to a user, for example.

The performance power curve may characterize both aerobic and anaerobic performance of the user. In order to acquire an accurate estimate of the aerobic and anaerobic performance of the user, the time series data preferably includes data from both kinds of athletic performances. In an embodiment, the first duration is smaller than a crossover time representing a time for maintaining maximal aerobic speed (MAS), and the second duration is greater than the crossover time. As described earlier, the crossover time t_{c} may be 5 to 7 minutes, or in an embodiment, t_{c} is 6 minutes.

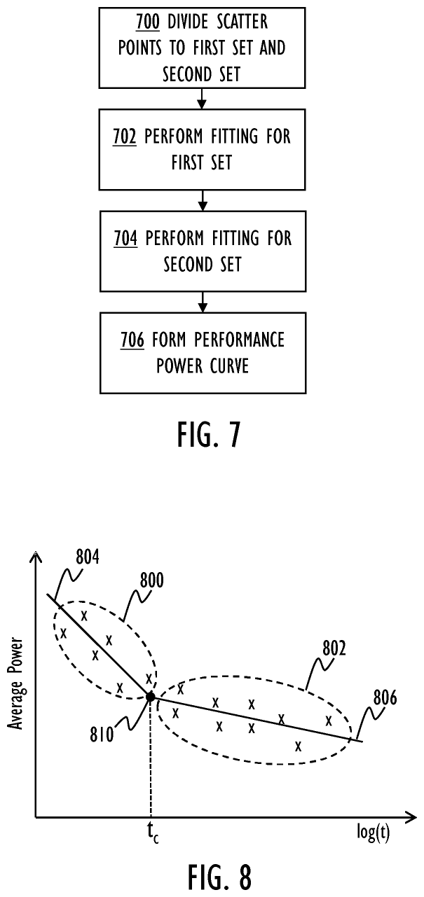

In an embodiment illustrated in FIG. 7 and FIG. 8, the apparatus is configured to divide 700 the set of scatter points to a first set of scatter points 800 and a second set of scatter points 802, wherein durations of the first set of scatter points are smaller than or equal to a cut-off time, and durations of the second set of scatter points are greater than or equal to the cut-off time; and perform 702 the fitting for the first set of scatter points to obtain a first piece 804 of the performance power curve, and perform 704 the fitting for the second set of scatter points to obtain a second piece 806 of the performance power curve, and form 706 the performance power curve on the basis of the first piece of the performance power curve and the second piece of the performance power curve. It has been observed that the performance power curve may be best fit in 2 pieces. The performance power curve may thus be a (2-piece) piecewise function. In an embodiment, the cut-off time is the crossover time (t_{c}). Optimal fitting may be achieved when the performance power curve is a piecewise function with a breakpoint 810 at t_{c}. The apparatus may perform the fitting such that the breakpoint is fixed at the crossover time. Such a fitting separates aerobic and anaerobic performances to the first and second pieces of the performance power curve, respectively, and allows for more accurate characterization of the user's performance as individuals may have drastically different aerobic and anaerobic performance characteristics. Athletic performances with a duration smaller than or equal to t_{c} may be predominantly anaerobic or at least representative of anaerobic performances, and athletic performances with a duration greater than or equal to t_{c} may be predominantly aerobic or at least representative of aerobic performances.

In FIG. 8 the duration is expressed on a logarithmic scale. This facilitates advantages regarding the fitting and the form of the performance power curve. In an embodiment, the performance power curve is a piecewise linear function. Fitting of linear functions is computationally simple and possibly more accurate that fitting more complex functions. The performance profile curve may be defined by parameters such as the respective slopes E, F, of pieces 804 and 806 and the breakpoint 810. The breakpoint may correspond to duration t_{c}.

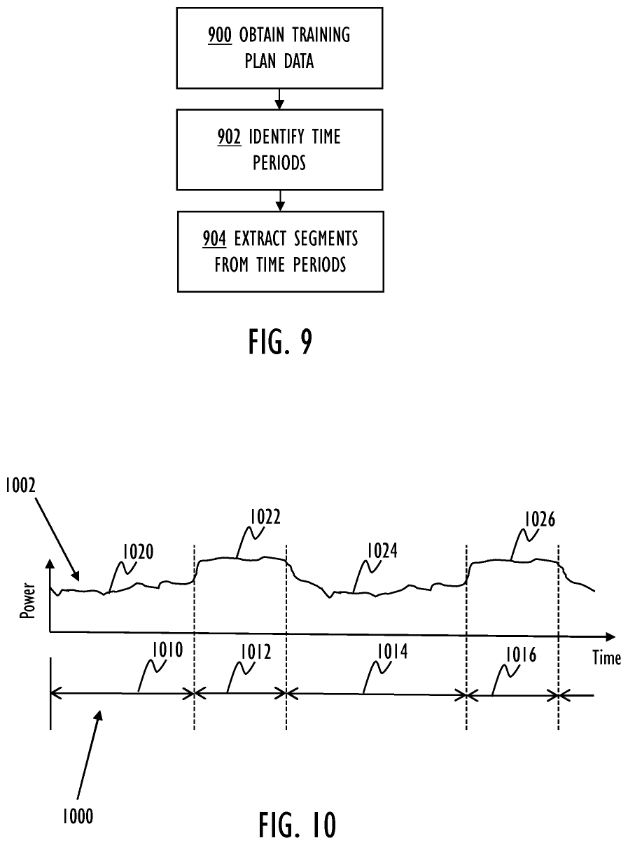

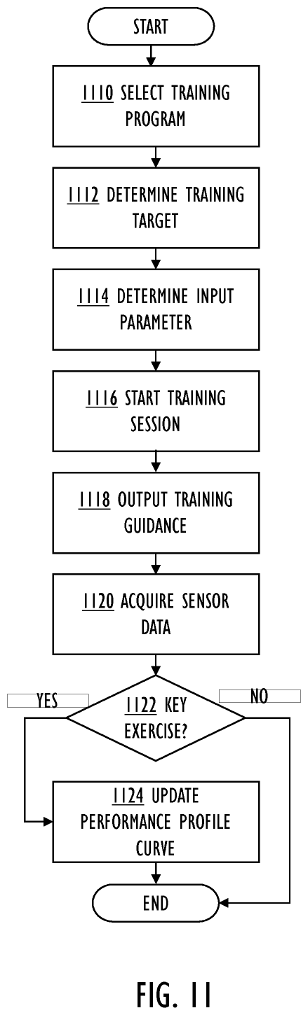

FIG. 9 and FIG. 10 illustrate embodiments related to acquisition of the time series data. In an embodiment, the apparatus is configured to obtain 900 training plan data 1000 associated with the time series data 1002; identify 902, on the basis of the training plan data, one or more time periods of the time series data that have training intensities greater than an inclusion threshold; and extract 904 the plurality of segments from the time series data of the identified one or more time periods. The training plan data may comprise training program and/or training session plan data. The training program may comprise a long-term plan that spans over several days, weeks or months. For example, a training program that follows a 3 week cycle may comprise the following entries: 1) Productive week (2 key exercises), 2) Productive week (1 key exercise), and 3) Recovery week. Key exercises may refer to high-intensity exercises that are expected to produce time series data that is suitable for the purposes described herein. The apparatus may be configured to identify the one or more time periods as one or more key exercises on the basis of the training plan data. The training intensities may be defined as number of training sessions per week, energy expenditure or calories burned, total or average power, total duration, or other types of data that may be measured by sensors described further in this document. The training intensities may refer to planned or measured training intensities. The inclusion threshold may be a value of one or more of the aforementioned types of data. The apparatus may or may not acquire the time series data of the entire one or more time periods; the apparatus may be configured to acquire (only) a part of the time series data of the identified one or more time periods.

The training session plan data may comprise information on training intervals and other time periods within a single training session. The training plan data of FIG. 10 illustrates intervals 1010, 1012, 1014 and 1016. The time series data segments corresponding to these intervals are 1020, 1022, 1024, 1026, respectively. Of these training interval periods, 1010 and 1014 may be low-intensity training interval periods, and 1012 and 1016 may be high-intensity interval periods. Information on the intensity of the training interval periods may be encoded within the training plan data. In an embodiment, the one or more time periods comprise one or more training interval periods 1012, 1016, and the apparatus is configured to extract one or more of the plurality of segments as the one or more training interval periods. The apparatus may thus extract time series data segments 1022 and 1026 corresponding to training interval periods 1012, 1016.

In an embodiment, the apparatus is configured to acquire time series data that represents power output of athletic performances of one or more users. The one or more users may include or exclude the user 100. When the one or more users does not include the user 100, the apparatus may provide the training instruction to the user 100 without having to acquire time series data from athletic performances of the user 100. The user therefore does not necessarily need to perform any athletic performances for the data acquisition, but still receives the training instruction from the apparatus.

FIG. 11 illustrates embodiments related to utilizing and updating the performance power curve during a training session. The apparatus may be configured to select 1110 a training program e.g. in response to a user input. The user may indicate that they wish to train e.g. for a marathon, and provide a user input to the apparatus selecting a corresponding training program e.g. from a selection of training programs displayed on the user interface. The apparatus may be configured to determine 1112, 1114 the training target and the input parameter e.g. on the basis of the training program by selecting the training target and duration and/or distance of a scheduled training session, for example. The apparatus may be configured to start 1116 the training session with the training target and input parameter specified above. During the training session, the apparatus may cause 1118 the user interface to output training guidance based on the set of training zones to the user. The apparatus may be configured to acquire 1120 sensor data or further time series data during the training session. The apparatus may determine 1122 whether the training session is/was a key exercise, and if so, the apparatus may be configured to extract training data of the training session and trigger update of the performance power curve. The apparatus may extract one or more segments from the sensor data or further time series data acquired during the training session and use that as the training data. Alternatively, the apparatus may take the time and duration of a work period or work periods of the training session (excluding warm-up and cooldown, if present) or the whole training session for the computation of the average power output of the training session. The apparatus may be configured to update 1124 the performance profile curve and the set training zones on the basis of the training data. The updating may be performed as in steps 204, 206, 208 and/or 210 of the method of FIG. 2, but using the segments extracted from the time series data acquired earlier and the sensor data acquired during the training session.

In general, the apparatus may acquire new training data after performing the fitting in 222 or 208; detect, from the new training data that the user's maximum power output has increased since performing the fitting (e.g. the user has made a new record time of crossing a certain distance); performing said fitting 208 or 222 again by using the new training data and determining 210 or 224 a new set of training power output zones on the basis of a new performance power curve resulting from the newly performed fitting, generating 212 or 226 a new training plan for a new physical exercise on the basis of the new set of training power output zones, and causing the user interface to output 214 or 228 the new training plan. In this manner, the procedure of FIG. 2A or 2B may be reiterated upon acquiring more training data and detecting a substantial change in the training data, crossing a certain triggering threshold.

One or more of the steps of the above embodiments may be combined with the method of FIG. 2A or 2B and/or its embodiments to provide a self-updating training guidance solution.

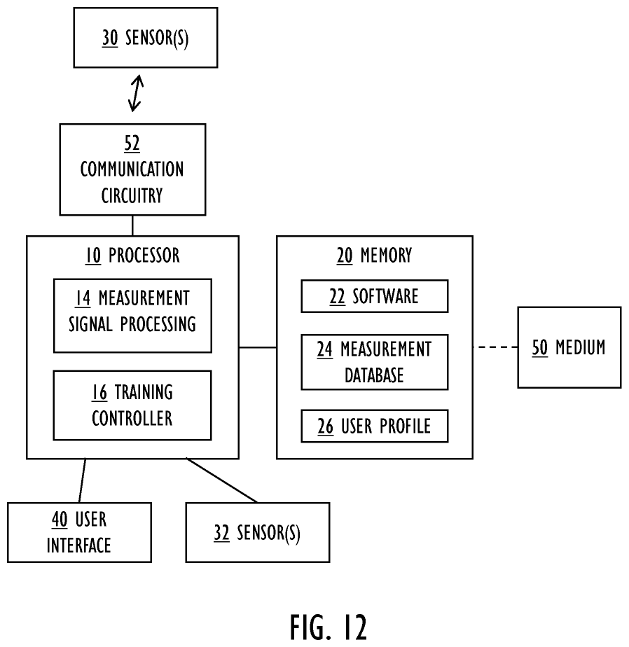

FIG. 12 illustrates embodiments of an apparatus or part of a system as a block diagram. The apparatus may be the wearable device 102, the portable electronic device 104, the wearable sensor device 106, or the server computer 108, for example. The apparatus of FIG. 12 may comprise a processing system configured to perform the method of FIG. 2A or 2B, or any one of the embodiments thereof. The processing system may comprise at least one processor 10 and at least one memory 20. The apparatus may further comprise a user interface 40 comprising a display screen or another display unit, an input device such as one or more buttons and/or a touch-sensitive surface, and an audio output device such as a loudspeaker. In some embodiments, the user interface 40 comprises a haptic output device configured to provide haptic indications to a user.

The processor 10 may comprise a measurement signal processing circuitry 14 configured to process the time series data by controlling at least some of the steps of the procedure of FIG. 2, such as steps 200, 202, 204, 206, and 208, or any one of the embodiments thereof. The measurement signal processing circuitry 14 may be configured to acquire time series data that represents power output of athletic performances from sensors 30 and/or from sensors 32 connected to the processor 10. The processor 10 may further comprise a training controller (circuitry) 16 configured to control training-related steps of the procedure of FIG. 2, such as generating 212 and outputting 214 the training instruction. The measurement signal processing circuitry 14 or the training controller 16 may be configured to determine 210 the training zones, depending on the implementation.

The apparatus may comprise a communication circuitry 52 connected to the processor 10. The communication circuitry 52 may comprise hardware and software suitable for supporting e.g. Bluetooth® communication protocol such as Bluetooth Smart specifications. It should be appreciated that other communication protocols are equivalent solutions as long as they are suitable for a personal area network (PAN) or suitable for measurement scenarios described in this document. The communication circuitry may comprise a radio modem and appropriate radio circuitries for establishing a communication connection between a server computer and a wearable device. Suitable radio protocols may include IEEE 802.11-based protocols or cellular communication protocols. The processor 10 may use the communication circuitry 52 to transmit and receive frames or data according to the supported wireless communication protocol. The frames may carry a payload data comprising the time series data measured by the sensors 30. In some embodiments, the processor 10 may use the communication circuitry 52 to transmit the time series data, segment power values, set of scatter points, performance power curve, set of training zones, and/or training instruction to another apparatus, e.g. to the server computer or the wearable device.

In the case of the distributed apparatus or system, a first apparatus such as the server computer may be configured to perform some steps of the method of FIG. 2, such as steps 200, 202, 204, 206, and 210. A second apparatus, such as the wearable device may be configured to perform the steps 212 and 214, for example. The first apparatus may transmit the outputs of one or more steps, such those of step 210 (the set of training zones) to the second apparatus to facilitate subsequent processing of the outputs by the second apparatus. Referring now to FIG. 11, in another example, the server computer may perform steps 1110, 1112, 1114, 1122, and 1124, and the wearable device may perform steps 1116, 1118, and 1120. The memory 20 may store a computer program product 22 defining computer program instructions for carrying out the method of FIG. 2A or 2B or any one of the embodiments thereof. The memory may further store a measurement database 24 comprising the measured the time series data, and/or data associated with and/or derived from the time series data, including e.g. the segment power values, set of scatter points, performance power curve, set of training zones, and/or the training instruction. The memory may further store a user profile 26 storing personal characteristics of the user, e.g. age, weight, etc.

The time series data may include data measured from the user by one or more sensors, such as sensors 30 and 32. The sensors 30 and/or 32 may be configured to measure the time series data from the user. The apparatus may comprise the sensors 30 and/or 32. The sensors may comprise one or more of the following: one or more heart activity sensors, one or more motion sensors, one or more location sensors, one or more barometric sensors, one or more swimming sensors, one or more power sensors, one or more bike sensors, and/or one or more temperature sensors.

The heart activity sensors may be configured to determine heart activity, such as heart rate, heart beat interval (HBI) and/or heart rate variability (HRV), for example. The heart activity sensors include, but are not limited to, a cardiovascular sensor (such as an ECG sensor), an optical heart activity sensor such as a photoplethysmography (PPG) sensor, or a bioimpedance plethysmography. The optical heart activity sensor may detect the heart activity of the user by optical heart rate measurement, which may comprise sending a light beam towards skin of the user and measuring the bounced and/or emitted light from the skin of the user. The light beam may alter when travelling through veins of the user and the alterations may be detected by the optical heart rate activity sensor. An embodiment of an ECG sensor is a wearable sensor device 106, such as a heart rate monitor belt illustrated in FIG. 1, for example. A PPG sensor may be integrated to a wearable device 102, such as a wrist device illustrated in FIG. 1, for example. Further, besides these types of heart activity sensors, other types of biosignal measurement sensors may be embedded into the heart activity sensors. These types include but are not limited to the following: a Laser Doppler-based blood flow sensor, a magnetic blood flow sensor, an electromechanical film (EMFi) pulse sensor, a polarization blood flow sensor. In an embodiment, the heart activity sensor may produce raw sensor data of the heart activity and/or it may process the sensor data into heart activity information, such as heart rate data, for example.

Motion sensors may be configured to measure motion induced by the user to the motion sensors by moving their hands, chest, head, or other body parts to which the motion sensor attached to. The motion sensor may use other motion data, such as location data of the user, to determine motion of the user. In an example embodiment, the motion sensor comprises at least one of the following: an accelerometer, a magnetometer, and a gyroscope. The motion sensor may further comprise sensor fusion software for combining the accelerometer data and gyroscope data so as to provide physical quantities, such as acceleration data, velocity data, or limb trajectory data in a reference coordinate system having orientation defined by a predetermined gyroscope orientation.

Location sensors may utilize a global navigation satellite system (GNSS) or other satellite-based, or radio system-based system for locating the user and measuring various parameters (speed, distance, location, route, altitude, gradient) relating to the movement of the user. Indoor location may be detected via indoor location tracking methods, such as mapping techniques including measuring Earth's magnetic fields or radio frequency signals.

Barometric sensors or altimeters may measure altitude or gradient by measuring atmospheric pressure and matching the measured atmospheric pressure to an altitude.

Swimming sensors may measure swimming specific parameters such as number of strokes or distance, for example.

Power sensors may measure power in various sports. Examples of power sensors include bike power sensors that may be fitted to a bike of the user, and running power sensors, such as footpods.

Bike sensors may be sensors attached to various parts of a (stationary) bike for measuring speed, cadence, or power (output), for example.

The time series data measured by the sensors, or determined by the apparatus on the basis of the time series data, may comprise: heart rate zones, heart rate samples, heart rate variation samples, heart beat interval samples, fat consumption rate, calorie consumption rate, consumed amount of calories, activity zones, activity samples, speed and/or pace samples, power samples, cadence samples, altitude samples, gradient samples, temperature samples, location samples, distance elapsed, time elapsed, pedal index, left-right balance, running index, training load, galvanic skin response samples, fluid balance, skin temperature samples, heading samples and/or bike angles.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware. The term ‘circuitry’ would also cover, for example and if applicable to the particular element, a baseband integrated circuit or applications processor integrated circuit for a mobile phone or a similar integrated circuit in a server, a cellular network device, or another network device.

In an embodiment, at least some of the processes described in connection with FIG. 2A or 2B may be carried out by a system comprising corresponding means for carrying out at least some of the described processes. Some example means for carrying out the processes may include at least one of the following: detector, processor (including dual-core and multiple-core processors), digital signal processor, controller, receiver, transmitter, encoder, decoder, memory, RAM, ROM, software, firmware, display, user interface, display circuitry, user interface circuitry, user interface software, display software, circuit, and circuitry. In an embodiment, the at least one processor, the memory, and the computer program code form processing means or comprises one or more computer program code portions for carrying out one or more operations according to any one of the embodiments of FIG. 2A or 2B or operations thereof.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, other electronic units designed to perform the functions described herein, or a combination thereof. For firmware or software, the implementation can be carried out through modules of at least one chipset (e.g, procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

Embodiments as described may also be carried out in the form of a computer process defined by a computer program or portions thereof.

Embodiments of the methods described in connection with FIG. 2A or 2B may be carried out by executing at least one portion of a computer program comprising corresponding instructions. The computer program may be in source code form, object code form, or in some intermediate form, and it may be stored in some sort of carrier, which may be any entity or device capable of carrying the program. For example, the computer program may be stored on a computer program distribution medium 50 readable by a computer or a processor. The computer program medium may be, for example but not limited to, a record medium, computer memory, read-only memory, electrical carrier signal, telecommunications signal, and software distribution package, for example. The computer program medium may be a non-transitory medium. Coding of software for carrying out the embodiments as shown and described is well within the scope of a person of ordinary skill in the art.

Even though the invention has been described with reference to one or more embodiments according to the accompanying drawings, it is clear that the invention is not restricted thereto but may be modified in several ways within the scope of the appended claims. All words and expressions should be interpreted broadly, and they are intended to illustrate, not to restrict, the embodiments. It will be obvious to a person skilled in the art that, as technology advances, the inventive concept may be implemented in various ways.

What is claimed is:

1. An apparatus comprising:
at least one processor; and
at least one memory storing a computer program code configured to cause the at least one processor to perform operations comprising:
acquiring training data that represents power output of a plurality of athletic performances of a user, the training data comprising first training data representing power output of a first athletic performance of a first duration and second training data representing power output of a second athletic performance of a second duration different from the first duration, and wherein the training data represents maximum power output of the user in the respective athletic performances;
fitting the training data into a decaying performance power curve representing power output as a function of duration of continuous athletic performance;
determining, on the basis of the fitting, a set of training power output zones that conform to the performance power curve by decaying as a function of the duration;
generating a training plan for a physical exercise on the basis of the set of training power output zones; and
causing a user interface to output the training plan to the user.

2. (canceled)

3. The apparatus of claim 12, wherein the maximum power output is defined in terms of a record average power output the user has performed.

4. The apparatus of claim 12, wherein the apparatus is configured to perform operations comprising:
acquiring new training data after performing the fitting;
detecting, from the new training data that the user's maximum power output has increased since performing the fitting; and
performing said fitting again by using the new training data and determining a new set of training power output zones on the basis of a new performance power curve resulting from the newly performed fitting; and
generating a new training plan for a new physical exercise on the basis of the new set of training power output zones and causing the user interface to output the new training plan.

5. The apparatus of any 1, wherein the training data comprises measurement data of at least the first athletic performance stored in the at least one memory, and wherein the at least one processor is configured to compute a power output of the first athletic performance on the basis of the measurement data.

6. The apparatus of claim 5, wherein the at least one processor is configured to acquire the training data by performing operations comprising:
identifying a continuous time interval of the first athletic performance that is associated with a power output greater than an inclusion threshold; and
selecting the power output and the duration of the continuous time interval for the fitting and exclude a power output of at least one other time interval of the first athletic performance from the fitting.

7. The apparatus of claim 1, wherein the at least one processor is configured to perform operations comprising:
obtaining an input parameter representing duration of the physical exercise and a training target for the physical exercise;
selecting, on the basis of the training intensity target, a training power output zone from the set of training power output zones;
determining a power output target for the physical exercise on the basis of the input parameter and the selected training zone; and
causing the user interface to output the power output target.

8. The apparatus of claim 7, wherein the power output target comprises a heart rate target and/or a speed target.

9. The apparatus of claim 7, wherein the training target describes a training effect on the user sought from the physical exercise, wherein selectable training targets include at least ‘recovery’ and ‘maximum performance’.

10. The apparatus of claim 7, wherein the input parameter is a target duration or a target distance of the physical exercise.

11. A computer-implemented method comprising:
acquiring training data that represents power output of a plurality of athletic performances of a user, the training data comprising first training data representing power output of a first athletic performance of a first duration and second training data representing power output of a second athletic performance of a second duration different from the first duration, and wherein the training data represents maximum power output of the user in the respective athletic performances;
fitting the training data into a decaying performance power curve representing power output as a function of duration of continuous athletic performance;
determining, on the basis of the fitting, a set of training power output zones that conform to the performance power curve by decaying as a function of the duration;
generating a training plan for a physical exercise on the basis of the set of training power output zones; and
causing a user interface to output the training plan to the user.

12. (canceled)

13. The method of claim 11, wherein the maximum power output is defined in terms of a record average power output the user has performed.

14. The method of claim 11, further comprising:
acquiring new training data after performing the fitting;
detecting, from the new training data that the user's maximum power output has increased since performing the fitting; and
performing said fitting again by using the new training data and determining a new set of training power output zones on the basis of a new performance power curve resulting from the newly performed fitting; and
generating a new training plan for a new physical exercise on the basis of the new set of training power output zones and causing the user interface to output the new training plan.

15. The method of claim 11, wherein the training data comprises measurement data of at least the first athletic performance stored in the at least one memory, and wherein the at least one processor is configured to compute a power output of the first athletic performance on the basis of the measurement data.

16. The method of claim 15, wherein the training data is acquired by performing operations comprising:
identifying (902) a continuous time interval of the first athletic performance that is associated with a power output greater than an inclusion threshold; and
selecting the power output and the duration of the continuous time interval for the fitting and exclude a power output of at least one other time interval of the first athletic performance from the fitting.

17. The method of claim 11, further comprising:
obtaining (500, 502) an input parameter representing duration of the physical exercise and a training target for the physical exercise;
selecting (504), on the basis of the training intensity target, a training power output zone from the set of training power output zones;
determining (506) a power output target for the physical exercise on the basis of the input parameter and the selected training zone; and
causing (508) the user interface to output the power output target.

18. The method of claim 17, wherein the power output target comprises a heart rate target and/or a speed target.

19. The method of claim 17, wherein the training target describes a training effect on the user sought from the physical exercise, wherein selectable training targets include at least ‘recovery’ and ‘maximum performance’.

20. The method of any preceding claim 17, wherein the input parameter is a target duration or a target distance of the physical exercise.

21. A computer program product embodied on a non-transitory medium readable by a computer and comprising program instructions which, when executed by the computer, cause the computer to carry out a computer process comprising:
acquiring training data that represents power output of a plurality of athletic performances of a user, the training data comprising first training data representing power output of a first athletic performance of a first duration and second training data representing power output of a second athletic performance of a second duration different from the first duration, and wherein the training data represents maximum power output of the user in the respective athletic performances;
fitting the training data into a decaying performance power curve representing power output as a function of duration of continuous athletic performance;
determining, on the basis of the fitting, a set of training power output zones that conform to the performance power curve by decaying as a function of the duration;
generating a training plan for a physical exercise on the basis of the set of training power output zones; and
causing a user interface to output the training plan to the user.
